(19) United States
(12) Patent Application Publication
(10) Pub. No.: US 2023/0263998 A1
(43) Pub. Date: Aug. 24, 2023

(54) PROTECTIVE COVER

(71) Applicant: The Cleveland Clinic Foundation, Cleveland, OH (US)

(72) Inventors: Charles Martin, III, Pepper Pike, OH (US); Mark A. Miller, Bay Village, OH (US)

(21) Appl. No.: 18/111,138

(22) Filed: Feb. 17, 2023

Related U.S. Application Data
(60) Provisional application No. 63/319,923, filed on Mar. 15, 2022.
(60) Provisional application No. 63/311,800, filed on Feb. 18, 2022.

(51) Int. Cl. A61M 25/02; A61B 46/10; A61M 25/00

(52) U.S. Cl. CPC A61M 25/02; A61B 46/10; A61M 25/0009; A61M 2025/0253

(57) ABSTRACT

A medical assembly includes a sheath having a channel that extends therethrough; a medical device that extends into the channel of the sheath via a proximal end of the sheath; and a cover at least partially enclosing a space and having a first opening that provides access to the space, wherein the sheath extends through the first opening such that the medical device emerges from the proximal end of the sheath within the space.

### CROSS REFERENCE TO RELATED APPLICATIONS

This application claims priority to, and any other benefit of U.S. Provisional Application Nos. 63/311,800 (filed on Feb. 18, 2022) and 63/319,923 (filed on Mar. 15, 2022), the disclosures of which are incorporated herein by reference in their entirety.

### FIELD OF THE INVENTION

This application generally relates to a protective cover for a medical device, and more specifically, a protective cover for catheters or other medical devices carrying a biohazardous material.

### BACKGROUND

Catheters can be used for various medical procedures to deliver and/or extract material to/from a patient's body. However, such catheters may discharge hazardous materials as they are removed from the patient, thereby contaminating the surrounding environment.

For example, during a radioembolization (Y90) procedure, a catheter is used to inject a radioactive isotope (e.g., yttrium Y-90) in the form of isotope-embedded glass microspheres or resin beads into the liver to target cancer cells. Following injection, the catheter is removed from the patient with caution to avoid flicking radioactive material (e.g. the microspheres or beads) onto the patient, operator, floor, etc. Typically, gauze is used to cover the catheter tip to catch any such material as it exits the sheath. However, any error can lead to radioactive material contaminating the operating room, or a member of the treatment team, which can decommission the room for several hours or up to a day.

### SUMMARY OF THE INVENTION

In accordance with a first aspect, a medical assembly includes a sheath having a channel that extends therethrough; a medical device that extends into the channel of the sheath via a proximal end of the sheath; and a cover at least partially enclosing a space and having a first opening that provides access to the space, wherein the sheath extends through the first opening such that the medical device emerges from the proximal end of the sheath within the space.

In accordance with a second aspect, a method of forming a medical assembly includes folding a sheet assembly such that opposing sheet portions thereof are caused to overlie one another thereby defining a cover having a space therein accommodating a proximal end of a sheath within the space, such that the sheath extends outside the space through a first opening in the cover. The method further includes withdrawing a medical device from the sheath such that a distal end of the medical device emerges from the proximal end of the sheath within said space, wherein material eluted from the distal end of the medical device upon emerging from the sheath will be captured and retained within the space of the cover.

### BRIEF DESCRIPTION OF THE DRAWINGS

These and other features, aspects and advantages of the present disclosure can be further understood when read with reference to the accompanying drawings, wherein:

FIG. 1 is a front view of an example sheet assembly prior to conforming it into a protective cover for a catheter (or other medical device);

FIG. 2 is a back view of the sheet assembly;

FIG. 3 is a plan view of a medical assembly that includes the sheet assembly, a drape, a sheath, and a catheter;

FIG. 4 is a plan view of the medical assembly after the sheet assembly has been conformed into a protective cover for the catheter;

FIG. 5 is a cross-section view of the medical assembly taken along line 5-5 in FIG. 4;

FIG. 6 is a cross-section view of the medical assembly taken along line 6-6 in FIG. 4; and

FIG. 7 is a plan view of the medical assembly after the catheter has been retracted from the sheath.

### DETAILED DESCRIPTION

Relative directional terms herein such as “front,” “back,” “left,” “right,” “upper,” “lower,” and the like, are used for convenience when describing the embodiments as oriented in the drawings. Such orientation(s), however, are arbitrary, and different orientations can result rearranging which feature might be considered the “left” or “right,” etc.

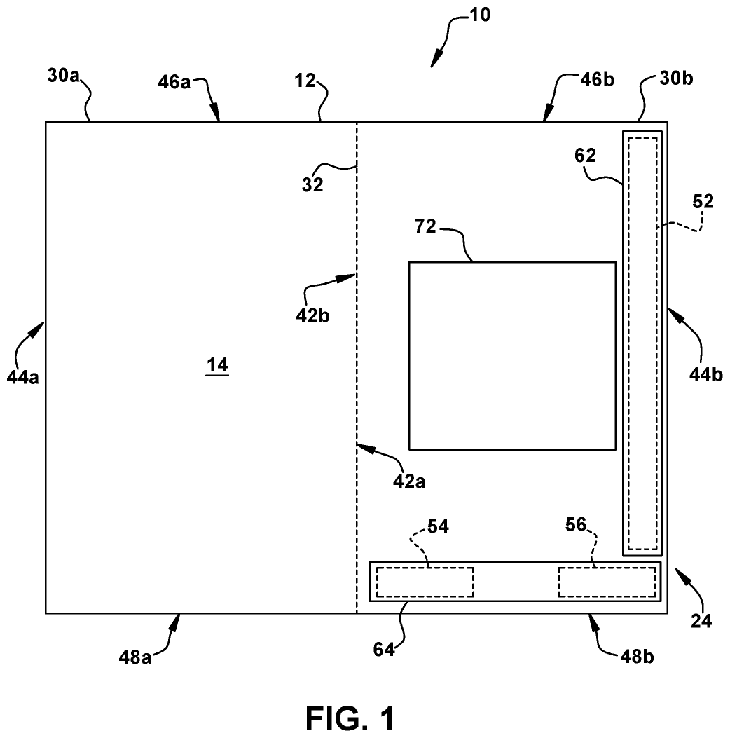

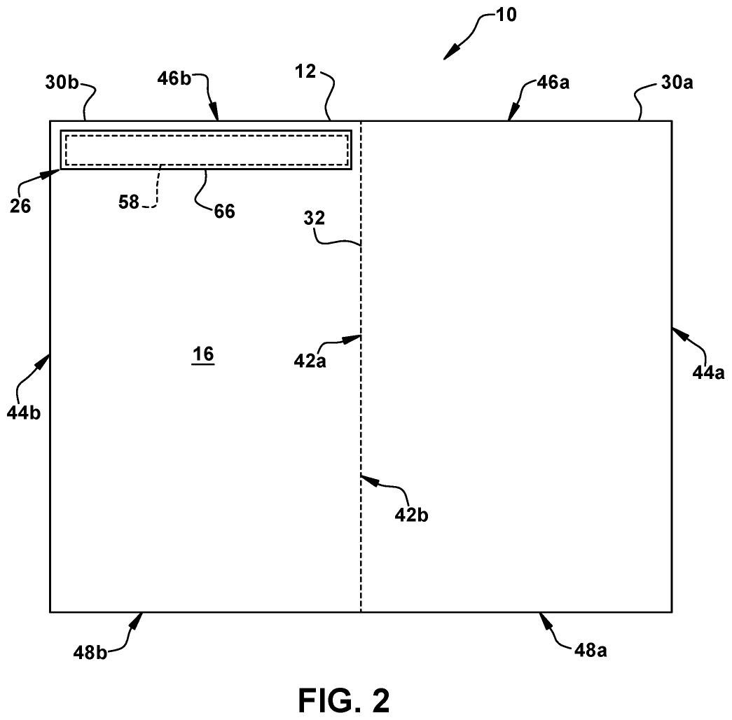

Turning to FIGS. 1 and 2, an example sheet assembly 10 that can be conformed (e.g. by folding) into a protective cover for a medical device (e.g., catheter) is shown. The sheet assembly 10 has a flexible sheet 12 of transparent or semi-transparent material (e.g., plastic) having a front surface 14 (see FIG. 1) and a back surface 16 (see FIG. 2). Moreover, the sheet assembly 10 has front and back adhesive systems 24, 26 provided on the front and back surfaces 14, 16 of the sheet 12, respectively.

The sheet 12 has a left sheet portion 30a and a right sheet portion 30b that are foldable relative to each other along a fold line 32 of the sheet 12. The fold line 32 can be an imaginary line. Alternatively, the fold line 32 can be a discrete feature such as, for example, a pre-formed crease in the sheet 12 or a continuous or broken line printed thereon.

In the illustrated example, the fold line 32 bisects the sheet 12 such that the sheet portions 30a, 30b are substantially rectangular and symmetric to each other about the fold line 32. Moreover, each sheet portion 30a, 30b has a medial edge 42a, 42b at the fold line 32, a lateral edge 44a, 44b opposite to the medial edge 42a, 42b, a distal edge 46a, 46b that extends between and connects distal ends of the medial 42a, 42b and lateral 44a, 44b edges, and a proximal edge 48a, 48b that extends between and connects proximal ends of the medial 42a, 42b and lateral 44a, 44b edges. The medial edges 42a, 42b of the sheet portions 30a, 30b intersect along the fold line 32 of the sheet 12.

However, it is to be appreciated that the configurations of the sheet portions 30a, 30b depend on the overall shape of the sheet 12, and that the location and orientation of the fold line 32 along which it is folded to conform to the protective cover can vary. For instance, the fold line 32 may be skewed in some examples, such that the sheet portions 30a, 30b are asymmetric about the fold line 32 and/or have non-rectangular shapes. Broadly speaking, the sheet 12 can embody any configuration of two sheet portions that are foldable along a fold line of the sheet 12 so that when folded one sheet portion will materially overlap the other.

Each adhesive system 24, 26 of the sheet assembly 10 includes one or more segments of adhesive provided on the sheet 12. For instance, the front adhesive system 24 (see FIG. 1) in the present embodiment has a plurality of adhesive segments 52, 54, 56 applied to the front surface 14 of the sheet 10, such that one segment 52 extends along and adjacent to the lateral edge 44b of the right sheet portion 30b, while the other two segments 54, 56 extend along and adjacent to the proximal edge 48b of the right sheet portion 30b. Meanwhile, the back adhesive system 26 (see FIG. 2) has a single adhesive segment 58 that is applied to the back surface 16 of the sheet 10 such that it extends along and adjacent to the distal edge 46b of the right sheet portion 30b. Each adhesive segment 52, 54, 56, 58 is a continuous, elongated flat strip of adhesive in the illustrated embodiment, applied by attaching, coating or printing an adhesive layer on the associated sheet surface, for example. However, the adhesive segments 52, 54, 56, 58 may possess other configurations such as, for example, a bead of adhesive.

Optionally, the sheet assembly 10 can further include one or more release liners that cover the adhesive segment(s) as-supplied and prior to use. For instance, the sheet assembly 10 in the present embodiment has a first release liner 62 (see FIG. 1) that covers the adhesive segment 52, and a second release liner 64 that covers the adhesive segments 54, 56. A third release liner 66 (see FIG. 2) covers the adhesive segment 58. The liners 62, 64, 66 are applied to their associated adhesive segments 52, 54, 56, 58, such that each segment is interposed between sheet portion 30b and its associated liner 62, 64, 66. Moreover, as discussed further below, the liners 62, 64, 66 can be peeled from their associated adhesive segments 52, 54, 56, 58 to expose those segments when conforming the sheet assembly to a protective cover for a medical device.

It is to be appreciated that the sheet assembly 10 can have any number of adhesive segments in a variety of locations and configurations, as well as associated release liners protecting them prior to use. Moreover, the sheet assembly 10 may have fewer or more adhesive systems than illustrated in the present embodiment. In one example, the sheet assembly 10 can have a single adhesive system with a single adhesive segment provided on the front surface 14 of the sheet 12.

The sheet assembly 10 can have one or more layers of gauze provided on the front surface 14 of the sheet 12. For instance, the present embodiment has a sheet of gauze 72 adhered to the right sheet portion 30b of the sheet 12, the sheet of gauze 72 being substantially square in shape. However, the sheet of gauze 72 may have other shapes and/or be adhered to the left sheet portion 30a in other examples. Moreover, the sheet assembly 10 may have additional or alternative gauze layers.

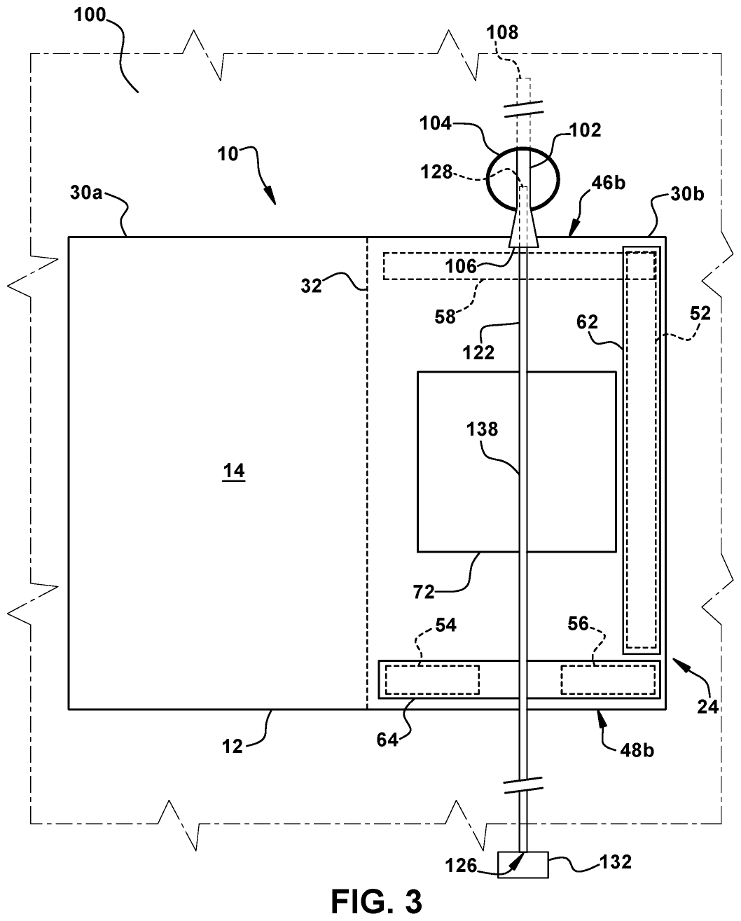

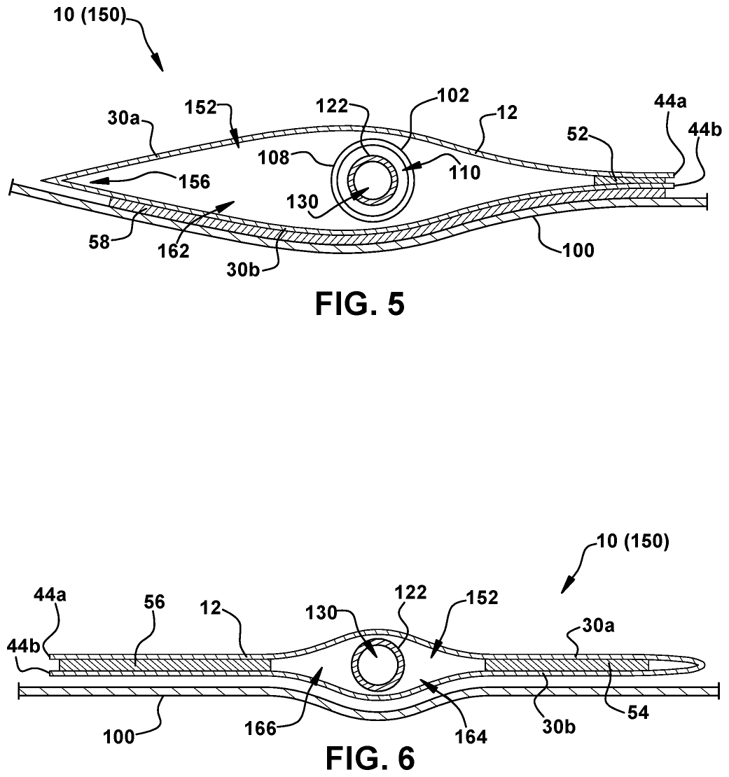

Turning to FIGS. 3-7, an example method of using the sheet assembly 10 in a medical procedure will now be described. As shown in the FIG. 3, a drape 100 can be placed over a patient's body, and a sheath 102 can extend through an aperture 104 in the drape 100 into the body. The sheath 102 is a tubular device having a proximal end 106 that resides outside of the body, a distal end 108 that resides within the body, and a channel 110 (see FIG. 5) that extends through the sheath 102 from its proximal end 106 to its distal end 108. The sheath 102 may also include a side tube (e.g. a lateral branch, not shown) that extends from its main body to another device (e.g., valve). The sheath 102 accommodates the passage for a medical device (e.g. a catheter) therethrough to remove material or deliver therapeutic or diagnostic agents, e.g. Y-90 impregnated microspheres.

For example, a catheter 122 can extend through the proximal end 106 of the sheath 102, and through its channel 110 so that it emerges from the distal end 108 within the patient. The catheter 122 also has a proximal end 126 that resides outside the body, a distal end 128 that can translate through the channel 110 of the sheath 102 into the patient's body, and a channel 130 (see FIG. 5) that extends through the catheter 122 from its proximal end 126 to its distal end 128. The catheter 122 in the present embodiment is a single-lumen catheter. However, the catheter 122 may have multiple lumens, branched lumens, or other configurations of one or more lumens.

The proximal end 126 of the catheter 122 can be fluidly coupled to another medical device 132 such as, for example, a source of therapeutic or diagnostic agent, or a vacuum source. In this manner, the catheter 122 can be used in a medical procedure to deliver material (e.g., radioactive isotopes) into the patient's body and/or extract material (e.g., bodily fluids) via its channel 130. However, as the catheter 122 is removed from the sheath 102, such material may be inadvertently released from the distal end 128 of the catheter 122, potentially contaminating the surrounding environment. Accordingly, as discussed below, the sheet assembly 10 can be conformed into a protective cover that can contain material released from the catheter 122.

The sheet assembly 10 can be initially arranged in an unfolded state as shown in FIG. 3, such that the distal edge 46b of the right sheet portion 30b is located proximate to the aperture 104 of the drape 100, and the right sheet portion 30b is arranged under the proximal end 106 of the sheath 102 as well as the catheter 122, above the drape 100. Moreover, the release liner 66 (see FIG. 2) covering the adhesive segment 58 on the back surface 16 of the right sheet portion 30b can be removed, so that the sheet assembly 10 can be affixed to the drape 100 via the adhesive segment 58.

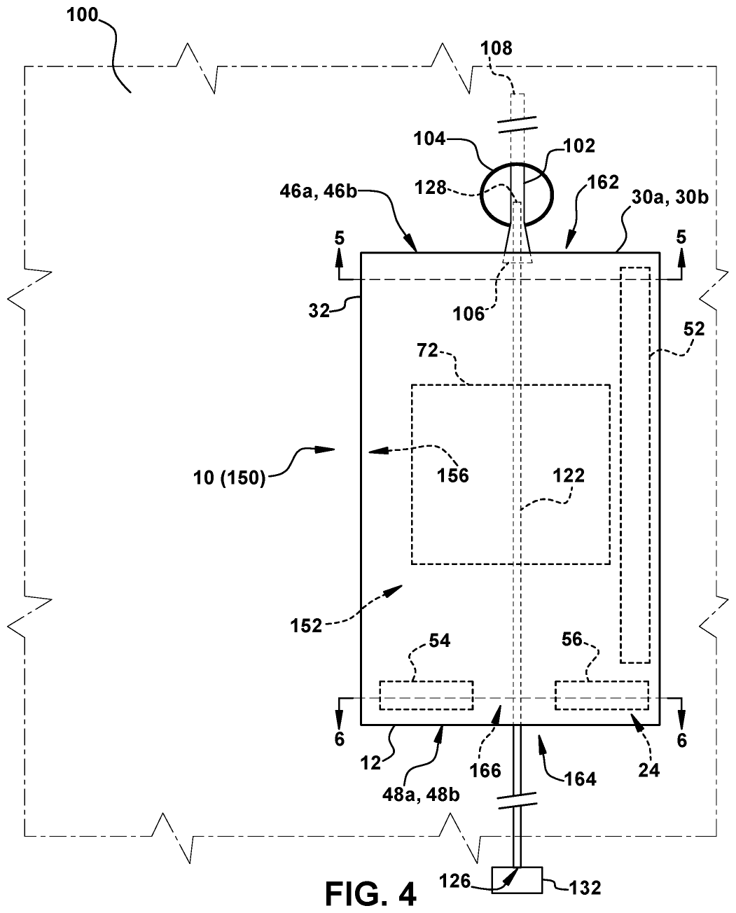

The release liners 62, 64 can then be removed, thereby exposing the underling adhesive segments 52, 54 and 56 on the front surface 14 of the right sheet portion 30b. Then the sheet 12 can be folded along the fold line 32 such that its left and right sheet portions 30a, 30b are bonded together by the adhesive segments 52, 54, 56 of the adhesive system 24, as shown in FIGS. 4-6. The sheet assembly 10 as configured will form a cover 150 defining a space 152 between the left and right sheet portions 30a, 30b. Moreover, the space 152 is partially circumscribed by an inner crease 156 of the sheet 12 (along the fold line 32) and the adhesive segments 52, 54, 56 of the adhesive system 24, thereby sealing the space 152 at those locations, and at least partially enclosing the space 152 between the opposing sheet portions 30a, 30b.

The cover 150 has distal and proximal openings 162, 164 for providing access to the space 152, the distal opening 162 being defined between the distal edges 46a, 46b of the sheet portions 30a, 30b and the proximal opening 164 being defined between the proximal edges 46a, 46b of the sheet portions 30a, 30b. Moreover, the cover 150 further defines a gap 166 between the adhesive segments 54, 56 on the surface to which those adhesive segments are applied. The gap 166 is located distal to the proximal opening 164 of the cover 150 and provides a confined opening into the space 152 between the adhesive segments 54, 56 when the opposing sheet portions 30a, 30b are adhered together via those adhesive segments 54, 56. Moreover, the space 152, openings 162, 164, and gap 166 are aligned such that the catheter 122 can extend along a substantially linear path therethrough.

Moreover, the sheath 102 will preferably extend through the distal opening 162 of the cover 150 into the space 152, such that its proximal end 106 is located within the space 152 and enveloped by the cover 150. That is, as shown in FIG. 5, the right and left (lower and upper, respectively, as seen in FIG. 5) sheet portions 30a, 30b of the cover 150 will substantially surround the proximal end 106 of the sheath 102.

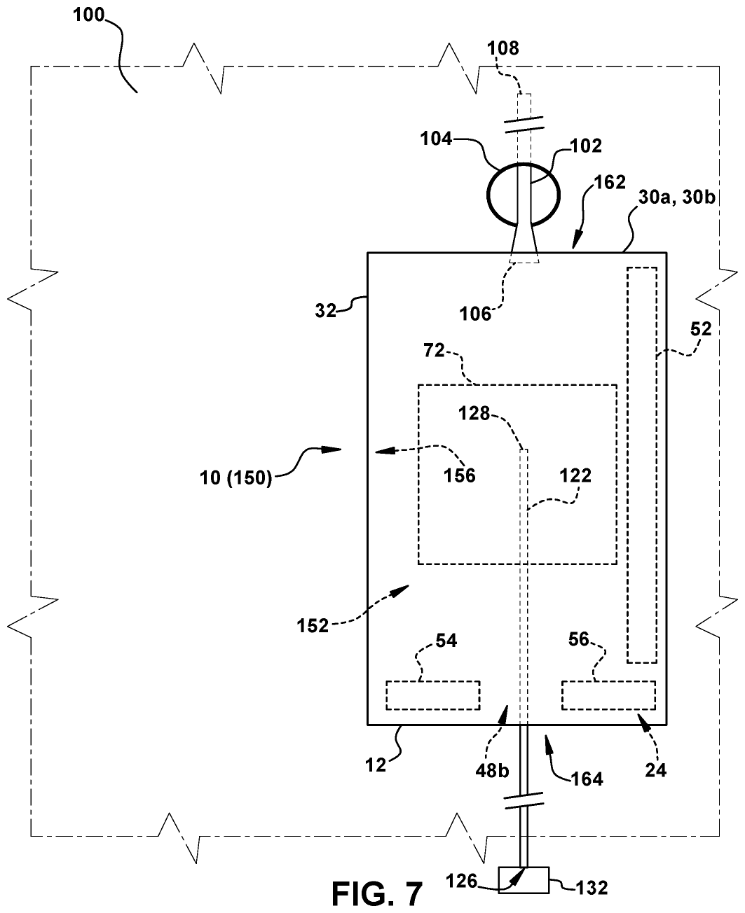

Once the cover 150 is formed from the sheet assembly 10, enveloping the proximal end 106 of the sheath 102, the catheter 122 can be retracted from the sheath 102 to the position shown in FIG. 7. That is, the catheter 122 can be translated relative to the sheath 102 and cover 150 until its distal end 114 emerges from the proximal end 106 of the sheath 102, within the space 152. During this translation, the catheter 122 will move through the space 152, openings 162, 164, and gap 166 of the cover 150. Moreover, because the proximal end 106 of the sheath 102 is located within the space 152 and enveloped by the cover 150, the distal end 114 of the catheter 122 will be similarly enveloped by the cover 150 and as it exits the sheath 102, thereby inhibiting material that may emerge from the distal end 114 from being expelled outside of the cover 150.

The catheter 122 will preferably be retracted until its distal end 114 is located above (i.e. registered with) the gauze layer 72. Notably, the transparency (or semi-transparency) of the sheet 12 can enable a user to view the catheter 122 as it is translated to ensure that the distal end 114 emerges from the sheath 102 and registers with the gauze layer 72. This way the gauze layer 72 can absorb and retain any material that elutes from the distal end 114 within the space 152. The catheter 122 and cover 150 can then be removed together as a unit from the drape 100 and the sheath 102, and transferred to, e.g., a biohazard waste container. During such transfer, the distal end 114 of the catheter 122 will preferably remain within the space 152 of the cover 150 and in-register with the gauze layer 72. Alternatively, the sheath 102 can be removed together with the catheter 122 and cover 150 such that its proximal end 106 remains within the space 152 during transfer.

The sheet assembly 10 as described above can thus be used in a medical procedure to prevent material that may be eluted from the catheter 122 upon withdrawal thereof from contaminating the exterior environment as it is removed from the sheath 102. This can be particularly advantageous during radioembolization or radioligand procedures, in which the catheter 122 is used to inject radioactive isotopes (e.g., yttrium Y-90) into the patient. However, the sheet assembly 10 may be used in other medical procedures as a cover for the catheter 122 or some other medical device (e.g., probe, endoscope, or some other tubular implement configured to deliver diagnostic or therapeutic agents to a patient) without departing from the scope of the disclosure.

Illustrative embodiments have been described, hereinabove. It will be apparent to those skilled in the art that various modifications and variations can be made without departing from the spirit and scope of the claimed invention. It is intended to include all such modifications and alterations within the scope of the present invention.

What is claimed is:

1. A medical assembly comprising:
a sheath having a channel that extends therethrough;
a medical device that extends into the channel of the sheath via a proximal end of the sheath; and
a cover at least partially enclosing a space and having a first opening that provides access to the space, wherein the sheath extends through the first opening such that the medical device emerges from the proximal end of the sheath within the space.

2. The medical assembly according to claim 1, the cover defining a second opening that provides access to the space, wherein the medical device extends through the second opening, into the space, and then into the channel of the sheath through the proximal end of the sheath.

3. The medical assembly according to claim 1, the cover comprising a sheet having a first sheet portion and a second sheet portion, wherein the sheet is folded along a fold line such that the first and second sheet portions substantially overlie one another to define the space therebetween.

4. The medical assembly according to claim 3, the cover comprising an adhesive system that bonds the first and second sheet portions together about a perimeter of the space.

5. The medical assembly according to claim 4, wherein the space is partially circumscribed by an inner crease of the sheet and the adhesive system.

6. The medical assembly according to claim 4, the adhesive system comprising a first adhesive segment and a second adhesive segment that define a gap therebetween, wherein the medical device extends through the gap into the space.

7. The medical assembly according to claim 4, the first sheet portion comprising a medial edge that is attached to the second sheet portion along the fold line, and a lateral edge opposite to the medial edge, wherein the adhesive system comprises a first adhesive segment that extends along and adjacent to the lateral edge.

8. The sheet assembly according to claim 7, the first sheet portion comprising a proximal edge that extends between a proximal end of the medial edge and a proximal end of the lateral edge, wherein the adhesive system further comprises one or more adhesive segments that extend along and adjacent to the proximal edge.

9. The medical assembly according to claim 3, the cover comprising a gauze layer within the space and attached to the sheet.

10. The medical assembly according to claim 3, wherein the sheet is transparent or semitransparent

11. The medical assembly according to claim 1, further comprising a drape having an aperture that the sheath extends therethrough, wherein the cover comprises an adhesive system that affixes the cover to the drape such that the first opening of the cover is disposed adjacent to the aperture in the drape.

12. The medical assembly according to claim 1, wherein the medical device is a catheter.

13. A method of forming a medical assembly, comprising:
folding a sheet assembly such that opposing sheet portions thereof are caused to overlie one another thereby defining a cover having a space therein accommodating a proximal end of a sheath within the space, such that the sheath extends outside the space through a first opening in the cover; and
withdrawing a medical device from the sheath such that a distal end of the medical device emerges from the proximal end of the sheath within said space, wherein material eluted from the distal end of the medical device upon emerging from the sheath will be captured and retained within the space of the cover.

14. The method according to claim 13, further comprising bonding together the opposing first and second sheet portions about a perimeter of said space via a first adhesive system provided on the sheet.

15. The method according to claim 13, wherein prior to folding the sheet, the method comprises arranging the sheet assembly over a drape such that an edge thereof adjacent to the first opening is disposed adjacent to an aperture in the drape.

16. The method according to claim 15, wherein prior to folding the sheet, the sheet assembly is arranged such that the sheet is interposed between the drape and the medical device.

17. The method according to claim 15, further comprising, prior to folding the sheet:
arranging the sheet assembly such that said edge of the sheet is located adjacent to the aperture, and then
bonding the sheet to the drape via an adhesive system provided on the sheet.

18. A method of confining material eluted from the medical device according to claim 1, comprising:
retracting the medical device from the channel of the sheath such that a distal end of the medical device emerges from the proximal end of the sheath within said space, wherein material eluted from the distal end of the medical device is at least partially confined within said space.

19. The method according to claim 18, the cover defining a second opening that provides access to the space, wherein the medical device is retracted such that it translates through the space and second opening.

20. The method according to claim 18, the cover comprising a gauze layer within the space, wherein the medical device is retracted until its distal end is located adjacent to the gauze layer.
